(19) United States
(12) Patent Application Publication
(10) Pub. No.: US 2026/0124263 A1
(43) Pub. Date: May 7, 2026

(54) Application, Extraction Method and Composition of Ganoderma lucidum Exosomes in Glioma Treatment

(71) Applicant: LIAOCHENG PEOPLE'S HOSPITAL, Liaocheng (CN)

(72) Inventors: Yilei XIAO, Liaocheng (CN); Bingwu YANG, Liaocheng (CN); Xiaofan XU, Liaocheng (CN); Lingyan DU, Liaocheng (CN); Ping WU, Liaocheng (CN); Yawen ZHANG, Liaocheng (CN); Xiaohui ZHANG, Liaocheng (CN); Hui YUE, Liaocheng (CN); Zongqiang XU, Liaocheng (CN); Lei WANG, Liaocheng (CN)

(21) Appl. No.: 19/380,613

(22) Filed: Nov. 5, 2025

(30) Foreign Application Priority Data
Nov. 5, 2024 (CN) 2024115621357

(51) Int. Cl. A61K 36/074; A61K 9/50; A61P 35/00

(52) U.S. Cl. CPC A61K 36/074; A61K 9/5063; A61P 35/00; A61K 2236/15; A61K 2236/19; A61K 2236/55

(57) ABSTRACT

This application relates to the technical field of biological products, and provides an application, extraction method and composition of TGL-ELNs in the treatment of glioma. The TGL-ELNs are applied in any one or more of the following aspects: a) Preparation of products for promoting apoptosis of glioma cells; b) Preparation of products for inhibiting proliferation of glioma cells; c) Preparation of products for inhibiting autophagy of glioma cells; d) Preparation of products for treating glioma. Verified through cell and animal experiments, the TGL-ELNs in this application can effectively induce apoptosis of glioma cells and inhibit autophagy of glioma cells to induce glioma cell death, and act on glioma cells through the AKT/mTOR signaling pathway and by inhibiting the production of autophagosomes in glioma cells.

### CROSS-REFERENCE TO RELATED APPLICATIONS

This application claims foreign priority of Chinese Patent Application No. 202411562135.7, filed on Nov. 5, 2024 in the China National Intellectual Property Administration, the disclosures of all of which are hereby incorporated by reference.

### Technical Field

This application relates to the technical field of biological products, and more specifically, to an application, extraction method and composition of TGL-ELNs □ Ganoderma lucidum (Taishan strain) exosomes □ in the treatment of glioma.

### Background Art

As a traditional Chinese medicinal material with a long history of medicinal use, the extraction and application of active ingredients of Ganoderma lucidum have always been a research focus. Ganoderma lucidum and its polysaccharides (peptides) can directly act on mononuclear macrophages, dendritic cells and cytotoxic T cells in the body, promote the expression of TNFα mRNA and IFNγ mRNA, increase the production of TNFα and IFNγ, and enhance the activity of cytokines. This further inhibits the immune escape of tumor cells, thereby inhibiting tumor cell proliferation, inducing tumor cell apoptosis, and ultimately killing tumor cells. Therefore, the anti-tumor effect of Ganoderma lucidum does not act directly on tumor cells, and its anti-tumor effect is relatively limited.

### SUMMARY OF THE INVENTION

The present application is provided to address the above-mentioned defects in the prior art. There is a need for an application, extraction method and composition of TGL-ELNs in glioma treatment. Verified through cell and animal experiments, the TGL-ELNs in this application can effectively induce glioma cell apoptosis, inhibit glioma cell autophagy, and induce glioma cell death. They act on glioma cells through the AKT/mTOR signaling pathway and by inhibiting the production of autophagosomes in glioma cells.

The first aspect of the present application provides an application of TGL-ELNs in any one or more of the following aspects:
a) Preparation of products for promoting apoptosis of glioma cells;
b) Preparation of products for inhibiting proliferation of glioma cells;
c) Preparation of products for inhibiting autophagy of glioma cells;
d) Preparation of products for glioma treatment.

The second aspect of the present application provides an extraction method for TGL-ELNs, which includes the following steps: (1) Grinding the soaked Ganoderma lucidum to obtain a grinding solution; (2) Performing coarse filtration on the grinding solution to obtain a coarse filtrate; (3) Mixing the supernatant of the coarse filtrate after centrifugation with an enzyme preparation for enzymatic hydrolysis to obtain an enzymatic hydrolysate; (4) Subjecting the enzymatic hydrolysate to impurity removal, filtration and concentration treatments to obtain TGL-ELNs.

The third aspect of the present application provides a composition. The active ingredients of the composition include at least TGL-ELNs, and the composition has any one or more of the following applications:
a) Promoting apoptosis of glioma cells;
b) Inhibiting proliferation of glioma cells;
c) Inhibiting autophagy of glioma cells;
d) Treating glioma.

The applications, extraction methods and compositions of TGL-ELNs in glioma treatment provided in each example of the present application have been verified by experiments. The results show that: TGL-ELNs can be taken up by glioma cells and enriched intracellularly; they can inhibit the proliferation of glioma cells, but have no proliferation-inhibiting effect on microglial cell lines and no obvious cytotoxicity to normal microglial cells; they have anti-glioma effect and the effect of inducing glioma cell apoptosis; they can effectively inhibit the occurrence of autophagy in glioma cells and further induce glioma cell death by means of the AKT/mTOR signaling pathway and inhibiting the production of autophagosomes in glioma cells, while showing no obvious cytotoxicity to normal microglial cells at the corresponding concentration; in addition, they also have good safety.

### BRIEF DESCRIPTION OF THE DRAWINGS

In the drawings that are not necessarily drawn to scale, the same reference numerals may describe similar components in different views. The same reference numerals with letter suffixes or different letter suffixes may represent different instances of similar components. The drawings generally illustrate various embodiments by way of illustration rather than limitation, and are used together with the specification and claims to explain the claimed embodiments. Where appropriate, the same reference numerals are used throughout all drawings to refer to the same or similar parts. Such embodiments are illustrative and are not intended to be exhaustive or exclusive embodiments of the device or method.

FIG. 1A shows the TEM of Taishan Ganoderma lucidum exosomes according to Example 1 of the present application;

FIG. 1B shows the NTA detection results diagram of Taishan Ganoderma lucidum exosomes according to Example 1 of the present application;

FIG. 2 shows that glioma cells treated with TGL-ELNs can be taken up into the cells by glioma cells according to Example 3 of the present application;

FIG. 3A shows the photos of glioma cells treated with Ganoderma lucidum exosomes and Ganoderma lucidum extract according to Example 4 of the present application;

FIG. 3B shows the cell proliferation result diagram after treating microglial cells/glioma cells with Ganoderma lucidum exosomes and Ganoderma lucidum extract according to Example 4 of the present application;

FIG. 4A shows the photos of the CDX model mouses according to Example 5 of the present application;

FIG. 4B shows the photos of dissected tumors from the glioma CDX model mouses according to Example 5 of the present application;

FIG. 4C shows the line chart of tumor volume in the glioma CDX model according to Example 5 of the present application;

FIG. 5 shows the schematic diagram of TGL-ELNs inducing glioma cell apoptosis according to Example 6 of the present application;

FIG. 6 shows the Western Blotting result display diagram that TGL-ELNs can inhibit the expression of TM9SF1 and suppress autophagy-related pathways according to Example 7 of the present application;

FIG. 7 shows the electron microscopy result display diagram that treating glioma cells with TGL-ELNs can inhibit the occurrence of autophagosomes according to Example 8 of the present application;

FIG. 8 shows the LC3-Cherry dual fluorescence result display diagram that treating glioma cells with TGL-ELNs can inhibit the occurrence of autophagosomes according to Example 9 of the present application;

FIG. 9 shows that treating glioma cells with TGL-ELNs did not cause significant pathological tissue abnormalities in the liver, kidney, heart, lung, brain, and spleen of mice according to Example 10 of the present application.

### SPECIFIC IMPLEMENTATION MODES

To enable those skilled in the art to better understand the technical solutions of the present application, the present application will be described in detail below with reference to the accompanying drawings and specific implementation modes. The embodiments of the present application will be described in further detail below with reference to the accompanying drawings and specific examples, but this is not intended to limit the present application.

The terms “first”, “second” and similar words used in the present application do not indicate any order, quantity, or importance, but are only used for distinction. Words such as “include” or “comprise” mean that the element before the word covers the elements listed after the word, and do not exclude the possibility of covering other elements.

According to the embodiments of the present application, an application of TGL-ELNs in any one or more of the following aspects is provided:
a) Preparation of products for promoting apoptosis of glioma cells;
b) Preparation of products for inhibiting proliferation of glioma cells;
c) Preparation of products for inhibiting autophagy of glioma cells;
d) Preparation of products for glioma treatment.

As an important bioactive substance in Ganoderma lucidum, TGL-ELNs are small vesicles secreted by cells, with a diameter usually ranging from 30-150 nm. Studies have shown that exosomes can carry a variety of specific substances such as proteins, RNA, and DNA, and exosomes play an important role in physiological processes such as regulating the balance of intracellular and extracellular environments, signal transduction, and immune response.

Experimental studies have found that exosomes can be taken up by glioma cells, have an anti-glioma cell effect, and have low toxicity to microglial cells.

In some embodiments, the application of preparing products for inhibiting autophagy of glioma cells at least exhibits the following: inhibiting the autophagic pathway through the AKT/mTOR signaling axis and inhibiting the production of autophagosomes in glioma cells. The autophagic pathway is inhibited through the AKT/mTOR signaling axis. Since the autophagic pathway is inhibited through the AKT/mTOR signaling axis, the production of autophagosomes in glioma cells can be further inhibited. In some embodiments, TGL-ELNs can act on autophagic pathway proteins including: LC3B1/2, AKT, P-AKT, mTOR, P-mTOR, p62 and/or beclin1, etc. TGL-ELNs can induce glioma cell apoptosis by inhibiting autophagic pathway proteins and autophagic pathway-related protein TM9SF1.

In some embodiments, the product is a food, health product, drug, or experimental reagent, where the experimental reagent is used for basic research.

In some embodiments, when the product is a drug, the drug further includes at least one pharmaceutically inactive ingredient. The pharmaceutically inactive ingredients may be carriers, excipients, diluents, etc. commonly used in pharmacy. Furthermore, according to conventional methods, it can be formulated into oral preparations (such as powders, granules, tablets, capsules, suspensions, emulsions, syrups, sprays), external preparations, suppositories, and sterile injectable solutions for use.

The pharmaceutically inactive ingredients such as carriers, excipients, and diluents that can be included are well-known in the field, and those of ordinary skill in the art can confirm that they meet clinical standards.

In some embodiments, the carriers, excipients, and diluents include but are not limited to lactose, glucose, sucrose, sorbitol, mannitol, xylitol, erythritol, maltitol, starch, acacia, alginates, gelatin, calcium phosphate, calcium silicate, cellulose, methylcellulose, microcrystalline cellulose, polyvinylpyrrolidone, water, methylparaben, propylparaben, talc, magnesium stearate, mineral oil, etc.

In some embodiments, the drug of the present invention can be administered into the body through known methods. For example, through systemic intravenous delivery. Alternatively, administration can be performed via intravenous, transdermal, intranasal, mucosal, or other delivery methods. Such administration can be carried out via a single dose or multiple doses. Those skilled in the art understand that the actual dose to be administered in the present invention can vary to a large extent depending on a variety of factors, such as target cells, biological type or its tissues, the general condition of the subject to be treated, administration route, administration method, and so on.

In some embodiments, the administration objects of the drug are humans and non-human mammals. The non-human mammals include rats, mice, rabbits, monkeys, Etc.

According to the embodiments of the present application, an extraction method for TGL-ELNs is further provided, which includes: (1) grinding the soaked Ganoderma lucidum to obtain a grinding solution; (2) performing coarse filtration on the grinding solution to obtain a coarse filtrate; (3) mixing the supernatant of the coarse filtrate after centrifugation with an enzyme preparation for enzymatic hydrolysis to obtain an enzymatic hydrolysate; (4) subjecting the enzymatic hydrolysate to impurity removal, filtration and concentration treatments to obtain TGL-ELNs.

In some embodiments, the soaking described in step (1) refers to soaking the Ganoderma lucidum in PBS buffer solution. In some embodiments, the Ganoderma lucidum can be cut into pieces first before soaking, and preferably the size of each Ganoderma lucidum piece is less than or equal to 1 cm³.

In some embodiments, the soaking time is 2-24 hours; preferably 6-8 hours.

In some embodiments, the enzyme preparation includes one or more of pectinase, cellulase, β-1, 3-glucanase and ligninase.

In some embodiments, the rotation speed of the grinding treatment is 1000-3000 rpm/min. In some embodiments, the grinding solution is subjected to coarse filtration using three-layer or two-layer gauze. The centrifugation speed of the coarse filtrate after coarse filtration is 800-2000 rpm, and the centrifugation time is 5-30 minutes.

In some embodiments, the mass of the enzyme preparation used for enzymatic hydrolysis is 0.1-3% of the mass of Ganoderma lucidum; the mass fractions of each component in the enzyme preparation are 10-30% for pectinase, 30-50% for cellulase, 5-15% for β-1, 3-glucanase, and 20-40% for ligninase, respectively.

In some embodiments, the enzymatic hydrolysate is subjected to impurity removal through sequential steps of freezing, centrifugation and filtration. In some embodiments, the filtration screen is 80-120 mesh. In some embodiments, the filtrate after impurity removal is concentrated by tangential flow filtration to obtain TGL-ELNs. In some embodiments, the filter pore size of tangential flow filtration is 30-1000 nm; preferably, the filter pore size of tangential flow filtration is 30-600 nm.

According to the embodiments of the present application, a composition is further provided. The active ingredients of the composition include at least TGL-ELNs, and the composition has any one or more of the following applications:
a) Promoting apoptosis of glioma cells;
b) Inhibiting proliferation of glioma cells;
c) Inhibiting autophagy of glioma cells;
d) Treating glioma.

In some embodiments, the concentration of TGL-ELNs in the composition is greater than or equal to 4×10⁹ Particles/mL. 4×10⁹ Particles/mL is equivalent to 4E+9 Particles/mL; when the concentration of TGL-ELNs in the composition reaches 4×10⁹ Particles/mL, it can significantly kill glioma cells. TGL-ELNs at a concentration of 4×10⁹ Particles/mL have no obvious cytotoxicity to microglial cells.

In other embodiments, the present invention further provides the application of the aforementioned TGL-ELNs or composition in the treatment of other tumors, or in the preparation of products for the treatment of other tumors.

The other tumors refer to benign or malignant tumors occurring in the breast; among them, benign tumors include breast fibroadenomas and breast hyperplasia nodules, and malignant tumors include breast cancer (including ductal carcinoma and lobular carcinoma).

### Example 1

A1. Clean fresh Ganoderma lucidum and perform preliminary disinfection by irradiation.
A2. Chop the cleaned fresh Ganoderma lucidum to obtain Ganoderma lucidum pieces smaller than 1 cm³;
A3. Soak the Ganoderma lucidum pieces obtained in Step A2 in PBS for 6 hours;
A4. Transfer the soaked Ganoderma lucidum (from Step A3) to a high-capacity blender for shearing, grinding, and for shearing and grinding treatment, with a rotation speed of 3000 rpm.
A5. Perform coarse filtration on the grinding solution obtained in Step A4 using three layers of sterile gauze.
A6. Transfer the coarse filtrate obtained in Step A5 to a 50 ml sterile centrifuge tube for centrifugation to remove impurities, with a centrifugation speed of 1000 rpm and a centrifugation time of 20 minutes.
A7. Add the supernatant obtained in Step A6 to a complex enzyme preparation (comprising 20% pectinase, 40% cellulase, 10% β-1, 3-glucanase, and 30% ligninase) at an amount constituting 0.1-0.3% of the mass of the original Ganoderma lucidum for enzymatic hydrolysis to obtain an enzymatic hydrolysate.

A8. Transfer the enzymatic hydrolysate obtained in Step A7 to freezing impurity removal equipment, and perform freeze centrifugation and filtration to remove impurities using a 100-mesh filter screen.

A9. Concentrate the filtrate obtained in Step A8 by tangential flow filtration to obtain TGL-ELNs, with a filter pore size of 30-600 nm.

A10. Perform irradiation sterilization on the TGL-ELNs obtained in Step A9.

### Example 2 Particle Size and Electron Microscopy Analysis of TGL-ELNs

### (1) Particle Size Analysis

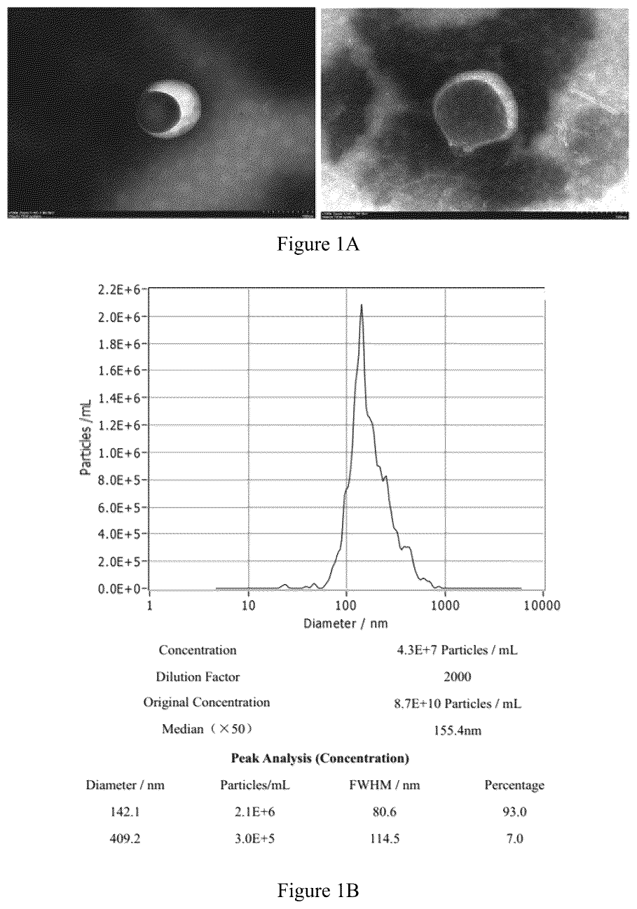

Add 1 mL of TGL-ELNs suspension (the composition of the suspension includes TGL-ELNs and PBS) into a sample cell, place the sample cell in a NanoSight (Nano-ZS) for testing and analysis, and draw a particle size spectrum. The particle size distribution diagram shown in Part of FIG. 1B is obtained, with the average diameter of TGL-ELNs being 155.4 nm.

### (2) Transmission Electron Microscopy (TEM) Analysis

For TEM imaging, add 30μL of TGL-ELNs suspension onto a discharge-treated sample grid, let it stand horizontally to allow TGL-ELNs adsorption, and then carefully blot the liquid dry with filter paper. Stain with 2% phosphotungstic acid dye for 2 minutes, and carefully blot the liquid dry with filter paper. After drying for 30 minutes, send the sample for analysis and take photos under TEM. The obtained transmission electron micrographs are shown in the two images of Part of FIG. 1A. The morphology consistent with TGL-ELNs can be observed from the two transmission electron micrographs in FIG. 1A.

### Example 3 Experiment for Detecting TGL-ELNs Uptake by Glioma Cells

Take glioma cell line U87 cells in the logarithmic growth phase, seed the cell suspension (containing MEM medium) into a Cell Carrier 96-well cell culture plate at a density of 2000 cells/well, and incubate overnight for 12 hours to allow cell adhesion, thereby obtaining the cultured cell suspension. According to the instructions of the PKH26 Dye Kit, use the PKH26 Dye Kit (Duolaimi, DL22065) to incubate TGL-ELNs at 37° C. for 37 minutes for staining treatment, and adjust the concentration of the stained TGL-ELNs to 4×10⁹ Particles/mL using MEM medium.

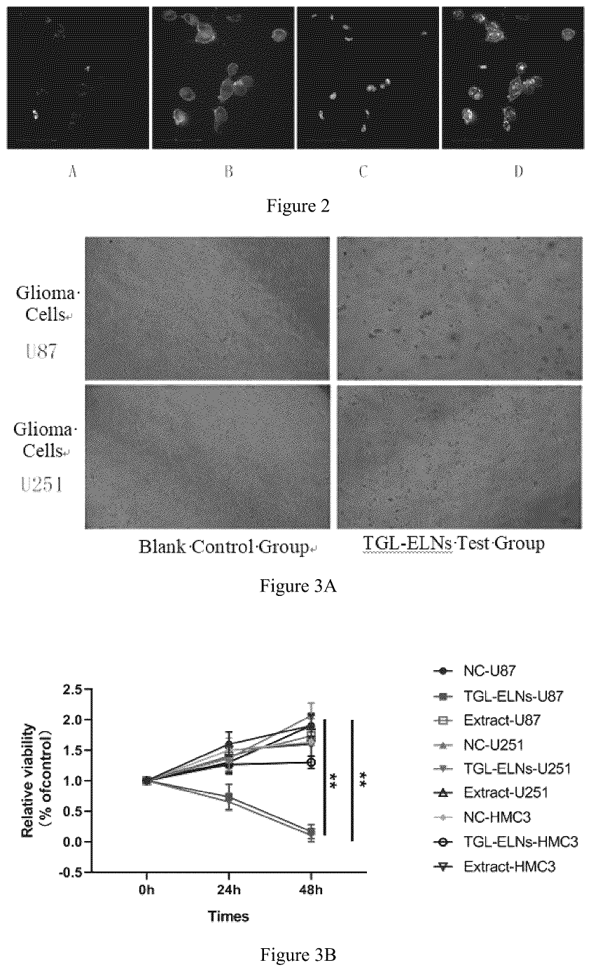

According to the instructions of the DiO Live Cell Dye (Biyuntian, C1993s) and Hoechst Nuclear Dye, incubate the above-mentioned cultured cell suspension at 37° C. for 15 minutes to stain the membrane structure and cell nucleus respectively. After staining the U87 cells with the cultured cell suspension, wash them twice with PBS, add the above-mentioned stained medium containing TGL-ELNs, and use a live cell workstation water lens to take photos of the TGL-ELNs channel, membrane channel, and nuclear channel respectively. The obtained images correspond to FIG. 2A, FIG. 2B, and FIG. 2C respectively, and the merged image is shown in FIG. 2D. In the treatment scheme involved in this application, the only experimental variable in the experimental group is the addition of TGL-ELNs. It can be seen from FIG. 2 that TGL-ELNs can be taken up by glioma cells into the cells and enriched intracellularly.

### Example 4 Detection of Anti-Glioma Cell Proliferation Effect of TGL-ELNs

Take glioma cell lines U87 and U251, and microglial cell line HMC3 in the logarithmic growth phase. Seed the cell suspension into a 96-well cell culture plate at a density of 6000 cells/well, and incubate overnight for 12 hours to allow cell adhesion. Set up three groups respectively: blank control group (adding culture medium to the 96-well cell culture plate with seeded glioma cells), TGL-ELNs test group (adding a mixture of TGL-ELNs suspension and culture medium to the 96-well cell culture plate with seeded glioma cells, where the volume ratio of TGL-ELNs suspension in the mixture is 10% and the concentration of TGL-ELNs suspension is 4×10⁹ Particles/mL), and Ganoderma lucidum extract treatment group (adding a mixture of Ganoderma lucidum extract and culture medium to the 96-well plate with seeded glioma cells, where the volume ratio of Ganoderma lucidum extract in the mixture is 10%). The preparation method of Ganoderma lucidum extract includes: first crushing 100 g of Ganoderma lucidum, then adding 100 mL of ultrapure water for soaking for 30 minutes; after soaking, heating to a water temperature of 100° C. and conducting a boiling water bath for 2 hours to obtain a mixture; filtering the mixture with 0.45μm and 0.22μm filters in sequence to obtain the Ganoderma lucidumextract. The aforementioned culture medium is MEM medium supplemented with 10% FBS. Set 9 replicates, with 5 wells as one group for parallel control; the volume of TGL-ELNs suspension added to each well of the TGL-ELNs test group is the same as the volume of Ganoderma lucidum extract added to each well of the Ganoderma lucidum extract treatment group. Continue culturing at 37° C., and conduct cell viability detection at 0 h, 24 h, and 48 h respectively. The detection method is as follows: add CCK8(10 μl/well), incubate at 37° C. for 4 hours, place in the dark for 30 minutes, and measure the OD450 absorbance value with a microplate reader to calculate cell viability. The results shown in FIG. 3B were obtained. The “relative cell survival rate” on the ordinate of FIG. 3B represents the survival ratio relative to the initial number of viable cells; the number of viable cells at 0 h is set to 1; after 24 h and 48 h, the ratio of “number of viable cells/initial number of viable cells” is the corresponding value on the ordinate. Among them, NC-U87 means the group containing only glioma cells U87 and culture medium; PELNs-U87 means the group where a mixture of TGL-ELNs suspension and culture medium is added to the well containing glioma cells U87 and culture medium; Extract-U87 means the group where a mixture of Ganoderma lucidum extract and culture medium is added to the well containing glioma cells U87 and culture medium; NC-U251 means the group containing only glioma cells U251 and culture medium; PELNs-U251 means the group where a mixture of TGL-ELNs suspension and culture medium is added to the well containing glioma cells U251 and culture medium; Extract-U251 means the group where a mixture of Ganoderma lucidum extract and culture medium is added to the well containing glioma cells U251 and culture medium; NC-HMC3 means the group containing only microglial cells HMC3 and culture medium; PELNs-HMC3 means the group where a mixture of TGL-ELNs suspension and culture medium is added to the well containing microglial cells HMC3 and culture medium; Extract-HMC3 means the group where a mixture of Ganoderma lucidum extract and culture medium is added to the well containing microglial cells HMC3 and culture medium.

From the photos on the left side of FIG. 3, it can be concluded that compared with the blank control group and the Ganoderma lucidum extract treatment group, the apoptosis rate of glioma cells (U87 and U251 cells) in the TGL-ELNs test group is significantly increased. From the relative cell activity curve in FIG. 3B, it can be observed that the relative cell activity of U87 and U251 cells decreases, while the relative cell activity of the microglial cell line HMC3 does not decrease. Moreover, the Ganoderma lucidum extract added in the same proportion does not show a glioma proliferation-inhibiting effect, indicating that the Ganoderma lucidum extract does not have an anti-glioma cell (U87 and U251 cells) effect.

### Example 5 Detection of In Vivo Anti-Glioma Effect of TGL-ELNs

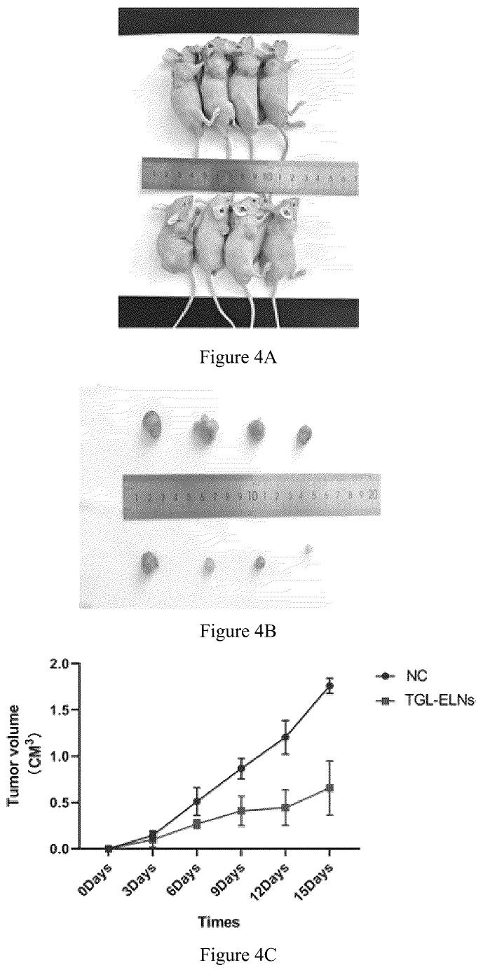

Four-week-old nude mice were divided into two groups with 4 mice in each group: the blank control group and the TGL-ELNs test group (200w cells (100μl) per mouse injected into the axillary region). A U87 cell glioma cell-derived xenograft (CDX) in vivo model was established. After tumor formation was confirmed, intratumoral injection of TGL-ELNs was initiated within 14 days of tumor formation at a dose of 1 μg/g every 48 h (1 μg of TGL-ELNs per 1 g of body weight every 48 h). The tumor size was measured and recorded every 3 days, as shown in Part C of FIG. 4. After 3-4 weeks, photos were taken, and the tumors were dissected and harvested, as shown in FIGS. 4A and 4B.

From FIG. 4, it can be concluded that the tumor volume of the TGL-ELNs test group (treated with intratumoral injection of TGL-ELNs) is significantly smaller than that of the blank control group during the administration of TGL-ELNs.

### Example 6 Detection of Glioma Cell Apoptosis Induced by TGL-ELNs

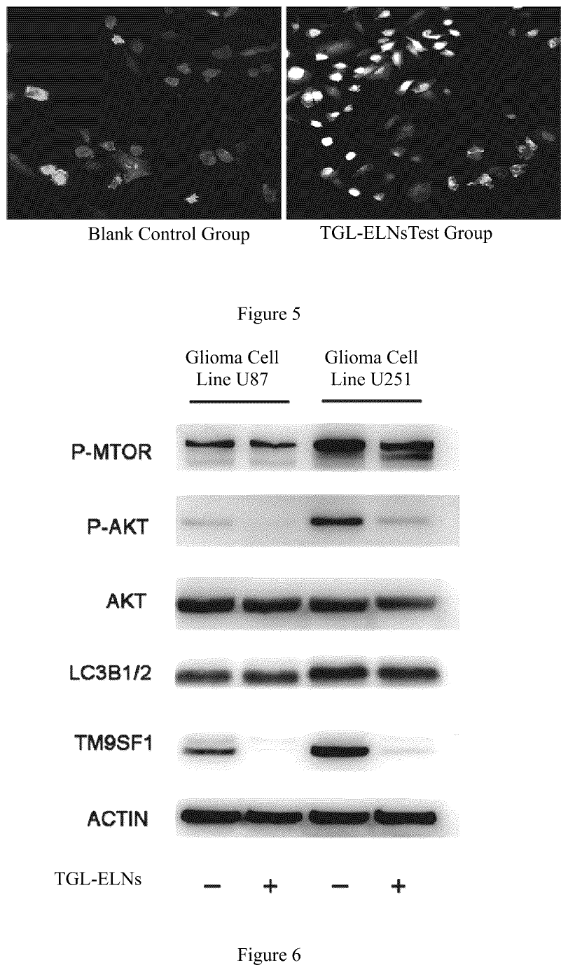

Take U87 cells of the glioma cell line in the logarithmic growth phase, and seed 200 μl of cell suspension into a 96-well cell culture plate at a density of 6000 cells/well, followed by overnight incubation for 12 h to allow cell adhesion. Set up the blank control group and the TGL-ELNs test group (inoculated with TGL-ELNs suspension at a concentration of 4×10⁹ Particles/mL) respectively. Continue culturing for 24 h, then add 5 μl of TUNEL staining solution to each well, incubate at 37° C. for 30 min, and use a live cell workstation to observe and photograph the cell apoptosis. The results shown in FIG. 5 were obtained. From FIG. 5, it can be concluded that the number of apoptotic cells in the TGL-ELNs test group is greater than that in the blank control group.

### Example 7 Detection of TGL-ELNs Inhibiting TM9SF1 Expression and Autophagy-Related Pathway Protein Expression

Take glioma cell lines U87 and U251 in the logarithmic growth phase, seed 2 ml of cell suspension into a 6-well cell culture plate at a density of 200,000 cells/well, and incubate overnight for 12 h to allow cell adhesion. Set up the blank control group and the TGL-ELNs test group (inoculated with TGL-ELNs suspension at a concentration of 4×10⁹ Particles/mL) respectively. Continue culturing for 24 h, then add protein lysis buffer to lyse the cells, centrifuge at 12,000 rpm for 10 minutes, and collect the supernatant;

Determine the protein concentration using a BCA assay kit;

Load 30 μg of total protein per well, add an appropriate amount of 6×protein loading buffer, and place in a boiling water bath for 10 minutes;

Prepare separating gel and stacking gel using a rapid PAGE gel kit:

Add an appropriate volume of protein sample, and add 6 μL of protein marker per well;

First, adjust the voltage to a constant 90V. When the bromophenol blue indicator of the electrophoretic sample passes through the separating gel, switch to a constant 110V for electrophoresis until the bromophenol blue indicator reaches the bottom of the separating gel;

Cut the PVDF membrane of appropriate size according to the number of samples and the molecular weight of the target protein, and soak it in methanol for about 5 seconds. Soak various materials in the electrophoresis transfer solution in the membrane transfer instrument, then stack them in the order of sponge pad-filter paper-PAGE gel-PVDF membrane-filter paper-sponge pad. After removing all air bubbles, perform membrane transfer at a constant current of 400 mA for 45 minutes;

After membrane transfer, take out the PVDF membrane and place it in 53% skimmed milk. Incubate on a shaker at room temperature with slow shaking for 1 hour for blocking;

Prepare primary antibodies with 3% skimmed milk, add them to the antibody incubation box, place the membrane with the front side up, and incubate at 4° C. overnight. After primary antibody incubation, wash the membrane with TBST 3 times, 5 minutes each time;

Incubate the membrane in secondary antibody at 37° C. for 30 minutes, then wash the membrane with TBST 3 times, 5 minutes each time;

Prepare ECL luminescent solution, drop it on the front side of the PVDF membrane, and perform development and fixation.

The results of Example 7 are shown in FIG. 6. In FIG. 6, P-MTOR (phosphorylated mammalian target of rapamycin), P-AKT (phosphorylated protein kinase B), AKT (protein kinase B), LC3B1/2(human microtubule-associated protein light chain 3II), TM9SF1(transmembrane 9 superfamily member 1), and ACTIN (internal reference protein) respectively represent autophagy pathway proteins or autophagy pathway-related proteins. From FIG. 6, it can be concluded that the TGL-ELNs test group can inhibit autophagy pathway proteins (LC3B1/2, P-AKT, AKT, P-mTOR) and autophagy pathway-related protein (TM9SF1) to induce glioma cell apoptosis. This indicates that the TGL-ELNs of the present application can act on glioma cells through the AKT/mTOR signaling pathway.

### Example 8 Observation of TGL-ELNs Inhibiting Autophagosome Formation by Electron Microscopy

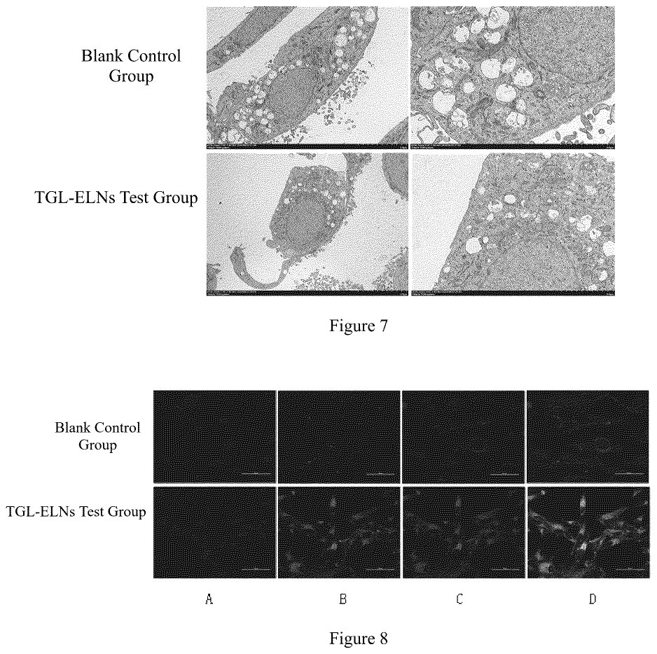

Take U87 cells of the glioma cell line in the logarithmic growth phase, seed 2 ml of cell suspension into a 6-well cell culture plate at a density of 20,000 cells/well, and incubate overnight for 12 hours to allow cell adhesion. Set up the blank control group and the TGL-ELNs test group (4×10⁹ Particles/mL) respectively, and continue culturing for 24 hours. Scrape the adherent cells and add electron microscopy fixative. After fixing the sample, add it to a discharge-treated sample grid, let it stand horizontally to allow TGL-ELNs adsorption, then carefully blot the liquid dry with filter paper. Stain with 2% phosphotungstic acid dye for 2 minutes, then carefully blot the liquid dry with filter paper again. After drying for 30 minutes, send the sample for analysis, observe and take photos using a transmission electron microscope (TEM), and obtain the transmission electron micrographs shown in FIG. 7.

### Example 9 Dual Fluorescence Detection of TGL-ELNs Inhibiting Autophagic Flux Using mRFP-cherry-LC3 (Autophagy Double-Labeled Adenovirus)

mRFP-cherry-LC3 is an LC3 protein (microtubule-associated protein light chain 3) labeled with mRFP fluorescent protein (monomeric red fluorescent protein) and cherry fluorescent protein. U87 cells were infected with mRFP-cherry-LC3 autophagy detection lentivirus, and a stable cell line was constructed. Seed cells into a CellCarrier 96-well plate at a density of 2000 cells/well, and incubate overnight for 12 hours to allow cell adhesion. Set up the blank control group and the TGL-ELNs test group (inoculated with TGL-ELNs suspension at a concentration of 4×10⁹ Particles/mL) respectively, and continue culturing for 24 hours. Add 5 μl of DAPI working solution to each well, and use a live cell workstation water lens to take photos: photos of the nuclear channel are shown in Group A of FIG. 8, photos of the mRFP fluorescent protein-autophagosome channel are shown in Group B of FIG. 8, and photos of the cherry fluorescent protein channel are shown in Group C of FIG. 8. Merge the images of the nuclear channel, mRFP fluorescent protein-autophagosome channel, and cherry fluorescent protein channel; the merged photos are shown in Group D of FIG. 8.

From FIGS. 7 and 8, it can be concluded that treatment with TGL-ELNs significantly reduces the number of autophagosomes in U87 cells. This suggests that TGL-ELNs can significantly inhibit the occurrence of autophagy, thereby inducing cell death.

### Example 10 Safety Detection of TGL-ELNs for In Vivo Anti-Glioma Effect

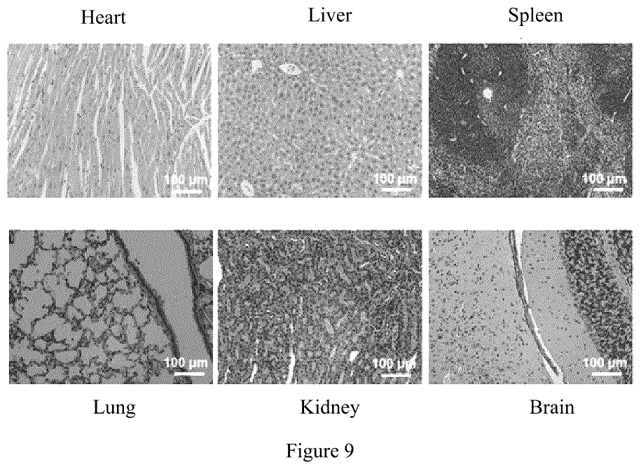

Four-week-old nude mice were divided into two groups with 6 mice in each group: the blank control group and the TGL-ELNs test group (200w cells (100 μl) per mouse injected into the axillary region). An in vivo cell-derived xenograft (CDX) model of U87 cell glioma was established. After tumor formation was confirmed, intratumoral injection of TGL-ELNs was initiated within 14 days of tumor formation at a dose of 1 μg/g every 48 h (1 μg of TGL-ELNs per 1 g of body weight every 48 h). After 3-4 weeks, photos were taken, and the mice were dissected to collect the liver, kidney, heart, lung, brain, and spleen for HE staining to evaluate histopathological changes. The results shown in FIG. 9 were obtained. It can be seen from FIG. 9 that the glioma treatment regimen using TGL-ELNs in this scheme did not cause significant pathological changes in the liver, kidney, heart, lung, brain, or spleen, indicating that the regimen has safety.

In addition, although exemplary embodiments have been described herein, their scope includes any and all embodiments based on the present application that include equivalent elements, modifications, omissions, combinations (e.g., schemes combining various embodiments), adaptations, or variations. Elements in the claims will be broadly construed based on the language used in the claims, and are not limited to the examples described in this specification or during the implementation of the present application; these examples will be construed as non-exclusive. Therefore, this specification and the examples are intended to be regarded merely as examples, and the true scope and spirit are indicated by the following claims and the full scope of their equivalents.

The above description is intended to be explanatory rather than restrictive. For example, the above-mentioned examples (or one or more aspects thereof) may be used in combination with each other. For instance, those of ordinary skill in the art may utilize other embodiments upon reading the above description. In addition, in the above detailed description, various features may be grouped together to simplify the present application. This shall not be construed as an intent that an unclaimed feature of the application is essential to any claim. On the contrary, the subject matter of the present application may include fewer than all the features of a specific embodiment of the application. Thus, the following claims are hereby incorporated into the detailed description as examples or embodiments, where each claim stands alone as a separate embodiment, and it is contemplated that these embodiments may be combined with each other in various combinations or arrangements. The scope of the present invention shall be determined with reference to the appended claims and the full scope of equivalents to which such claims are entitled.

The above embodiments are merely exemplary embodiments of the present application and are not intended to limit the present invention. The protection scope of the present invention is defined by the claims. Those skilled in the art may make various modifications or equivalent substitutions to the present invention within the spirit and scope of the present application, and such modifications or equivalent substitutions shall also be deemed to fall within the protection scope of the present invention.

1. An application of TGL-ELNs in any one or more of the following aspects:
a) Preparation of drugs for promoting apoptosis of glioma cells;
b) Preparation of drugs for inhibiting proliferation of glioma cells;
c) Preparation of drugs for inhibiting autophagy of glioma cells;
d) Preparation of drugs for treating glioma;
The extraction method of the TGL-ELNs comprises the following steps:
(1) Grinding the soaked Ganoderma lucidum to obtain a grinding solution;
(2) Performing coarse filtration on the grinding solution to obtain a coarse filtrate;
(3) Mixing the supernatant of the coarse filtrate after centrifugation with an enzyme preparation for enzymatic hydrolysis to obtain an enzymatic hydrolysate;
(4) Performing impurity removal, filtration and concentration treatments on the enzymatic hydrolysate to obtain TGL-ELNs.

2. The application according to claim 1, characterized in that, in the application of preparing drugs for inhibiting autophagy of glioma cells, the glioma treatment at least exhibits the following effects: inhibiting the autophagic pathway through the AKT/mTOR signaling axis and inhibiting the production of autophagosomes in glioma cells.

3. The application according to claim 1, characterized in that the drug further comprises at least one pharmaceutically inactive ingredient.

4. The application according to claim 1, characterized in that the administration objects of the drug are humans and non-human mammals.

5. The application according to claim 1, characterized in that, in step (1), the soaking refers to soaking Ganoderma lucidum in PBS buffer solution; the soaking time is 2-24 hours; the enzyme preparation in step (3) comprises one or more of pectinase, cellulase, β-1, 3-glucanase and ligninase.

6. The application according to claim 1, characterized in that, in step (3), the mass of the enzyme preparation used for enzymatic hydrolysis is 0.1-3% of the mass of Ganoderma lucidum; the mass fractions of each component in the enzyme preparation are respectively 10-30% for pectinase, 30-50% for cellulase, 5-15% for β-1, 3-glucanase, and 20-40% for ligninase.
